(19) United States
(12) Patent Application Publication
(10) Pub. No.: US 2024/0074716 A1
(43) Pub. Date: Mar. 7, 2024

(54) X-RAY DIAGNOSIS APPARATUS

(71) Applicant: Canon Medical Systems Corporation, Otawara-shi (JP)

(72) Inventors: Keisuke SUGAWARA, Otawara (JP); Saki HASHIMOTO, Nasushiobara (JP); Masanori MATSUMOTO, Nasushiobara (JP); Yoshiyuki SATO, Nasushiobara (JP); Hisayuki UEHARA, Otawara (JP); Shingo ABE, Nasushiobara (JP); Akio TETSUKA, Shioya (JP)

(73) Assignee: Canon Medical Systems Corporation, Otawara-shi (JP)

(21) Appl. No.: 18/461,556

(22) Filed: Sep. 6, 2023

(30) Foreign Application Priority Data
Sep. 6, 2022 (JP) 2022-141443

(51) Int. Cl. A61B 6/04; A61B 6/00

(52) U.S. Cl. CPC A61B 6/0407; A61B 6/461

(57) ABSTRACT

According to one embodiment, an X-ray diagnosis apparatus includes a table, an imaging unit, and processing circuitry. The table includes a table top on which a subject is placed. The imaging unit includes an X-ray tube which emits X-rays to the subject and an X-ray detector which detects the X-rays. The processing circuitry detects contact between an object and at least one of the imaging unit and the table. The processing circuitry executes control to display contact position information relating to a position in contact with the object based on a detection result.

### CROSS-REFERENCE TO RELATED APPLICATION

This application is based upon and claims the benefit of priority from Japanese Patent Application No. 2022-141443, filed Sep. 6, 2022, the entire contents of which are incorporated herein by reference.

### FIELD

Embodiments described herein relate generally to an X-ray diagnosis apparatus.

### BACKGROUND

A plurality of kinds of diagnosis instruments, therapeutic instruments, etc. are present in an examination room and an operation room. Therefore, during an examination or operation, instruments may interfere or collide with each other, or an instrument and a subject may interfere or collide with each other, depending on operation circumstances. Since an occurrence of such an interference or collision may lead to an accident, a health-care professional needs to carefully perform or prepare an examination or an operation so as to avoid the interference and collision. Under these circumstances, it is desirable to detect an interference or a collision or a prediction of occurrence of an interference or a collision, and to notify the health-care professional of the detection result.

### BRIEF DESCRIPTION OF THE DRAWINGS

FIG. 1 is a block diagram showing a configuration example of an X-ray diagnosis apparatus according to an embodiment.

FIG. 2 is a conceptual diagram showing an environment in which contact detection processing is executed in the X-ray diagnosis apparatus.

FIG. 3 is a flowchart showing an operation example of the X-ray diagnosis apparatus according to the embodiment.

FIG. 4 is a diagram showing an example of a placement of a contact detector.

FIG. 5 is a diagram showing a first display example of contact information.

FIG. 6 is a diagram showing a case of changing sensitivity in accordance with a contact region.

FIG. 7 is a diagram showing a second display example of the contact information.

### DETAILED DESCRIPTION

In general, according to one embodiment, an X-ray diagnosis apparatus includes a table, an imaging unit, and processing circuitry. The table includes a table top on which a subject is placed. The imaging unit includes an X-ray tube which emits X-rays to the subject and an X-ray detector which detects the X-rays. The processing circuitry detects contact between an object and at least one of the imaging unit and the table. The processing circuitry executes control to display contact position information relating to a position in contact with the object based on a detection result.

Now, the X-ray diagnosis apparatus according to the embodiment will be described with reference to the drawings. In the embodiment described below, elements assigned the same reference symbols are assumed to perform the same operations, and redundant descriptions thereof will be omitted as appropriate.

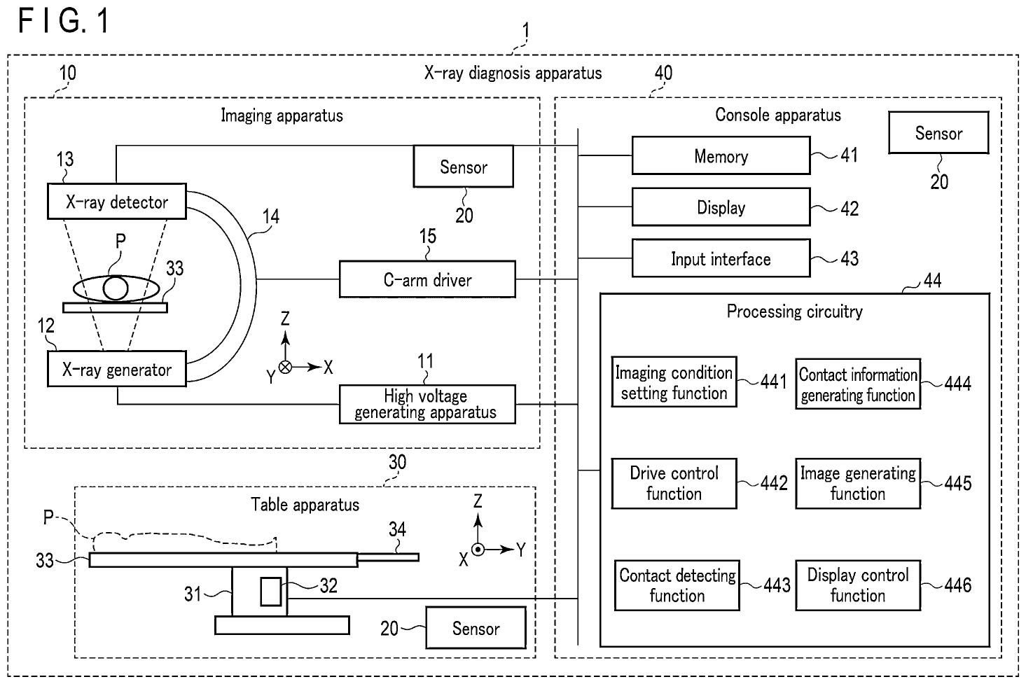

FIG. 1 is a block diagram showing a configuration example of an X-ray diagnosis apparatus 1 according to the embodiment. The X-ray diagnosis apparatus 1 is applicable to any type of apparatus, such as a general X-ray imaging apparatus, an X-ray fluoroscopic imaging apparatus, a cardiovascular imaging apparatus, a mammography apparatus, and the like. In the following, to make the explanation specific, the X-ray diagnosis apparatus 1 is assumed to be a C-arm X-ray fluoroscopic diagnosis apparatus. Thus, an “X-ray image” imaged by the X-ray diagnosis apparatus 1 means an X-ray fluoroscopic image. The X-ray diagnosis apparatus 1 includes an imaging apparatus 10, a table apparatus 30, and a console apparatus 40.

The imaging apparatus 10 is a module that performs X-ray fluoroscopic imaging for continuously or intermittently emitting a low dose of X-rays to a subject P. The imaging apparatus 10 may perform one-shot imaging (radioscopic imaging) by emitting a high dose of X-rays to the subject P. The imaging apparatus 10 includes a high voltage generating apparatus 11, an X-ray generator 12, an X-ray detector 13, a C-arm 14, a C-arm driver 15, and a sensor 20.

The high voltage generating apparatus 11 is an apparatus that applies a high voltage and supplies a filament current to an X-ray tube provided in the X-ray generator 12. Specifically, the high voltage generating apparatus 11 generates a high voltage to be applied between a cathode and an anode of the X-ray tube, and supplies the generated high voltage to the X-ray tube. The high voltage generating apparatus 11 may be either a transformer type or an inverter type.

The X-ray generator 12 is a mechanism that generates X-rays and adjusts a radiation quality and a dose of X-rays, and a size of a radiation field. The X-ray generator 12 includes the X-ray tube, and a filter and an X-ray diaphragm that adjust the X-rays.

The X-ray tube included in the X-ray generator 12 is a vacuum tube that generates X-rays. The X-ray tube is, for example, a rotating anode-type X-ray tube that generates X-rays by emitting thermal electrons toward a rotating anode. The X-ray tube has a tube bulb, and a filament (cathode) and a metal target (anode) provided in the tube bulb. The X-ray tube generates X-rays by accelerating the thermal electrons emitted from the filament (e.g. tungsten) with a high voltage, and causing the accelerated thermal electrons to collide with the metal target (e.g. tungsten, molybdenum, or copper). The X-ray tube emits X-rays to the subject P.

The filter included in the X-ray generator 12 adjusts the dose and radiation quality of the X-rays passing therethrough for the purpose of reducing the dose exposed to the subject P and improving the image quality of X-ray image data. The filter includes various types of X-ray filters, such as a beam filter, a dose lowering filter, a compensating filter, and the like. The various types of X-ray filters are interposed between the X-ray tube and the subject P.

The X-ray diaphragm provided in the X-ray generator 12 is a metal plate made of lead or the like. The X-ray diaphragm is provided in front of an X-ray emission window in the X-ray tube. The X-ray diaphragm is composed of four diaphragm blades each made of a metal plate of lead or the like. The diaphragm blades are driven by a driver (not shown) according to the region of interest of the subject P input by an operator via an input interface 43 of the console apparatus 40. The X-ray diaphragm adjusts a region in which the X-rays are blocked to a desired size by sliding the diaphragm blades by means of the driver.

The X-ray detector 13 is a mechanism that detects X-rays that have been emitted from the X-ray tube included in the X-ray generator 12 and have passed through the subject P. The X-ray detector 13 includes a flat panel detector (FPD), a gate driver, and a projection data generation circuit.

The FPD included in the X-ray detector 13 converts X-rays that have passed through the subject P to an electric charge and accumulates the electric charge. The FPD includes a plurality of minute semiconductor detection elements (pixels) arranged two dimensionally in rows and columns. Two types of semiconductor detection elements are known: a direct conversion type of converting X-rays directly to an electric charge; and an indirect conversion type of first converting X-rays by a fluorescent body to light and then converting the light to an electric charge. Either type may be used for the semiconductor detection elements of the embodiment. In the case of the former type, each semiconductor detection element of the direct conversion type includes a photoelectric film that generates electric charge in accordance with the amount of incident X-rays, a photodiode (PD) that accumulates the electric charge generated in the photoelectric film, an amplifier circuit that amplifies the electric charge, and an A/D converter that converts the amplified electric charge to a digital signal. The digital signal is sequentially read by drive pulses supplied from a gate driver. At this time, the digital signal is read out while the charge of the pixel corresponding to the digital signal is maintained.

The projection data generation circuit included in the X-ray detector 13 converts digital signals read from the FPD in parallel in units of rows or columns to time-series serial signals (projection data). The projection data generation circuit supplies the projection data to a memory 41 of the console apparatus 40. Thus, the X-ray detector 13 detects the X-rays emitted from the X-ray generator 12 pixel by pixel, and supplies the generated projection data to the memory 41.

The C-arm 14 retains the X-ray generator 12 and the X-ray detector 13 facing each other with the table top 33 of the table apparatus 30 interposed therebetween. The C-arm 14 is rotatable and slidable about each of a plurality of spatial axes. Accordingly, the C-arm 14 images the subject P on the table top 33 from a desired imaging direction.

The C-arm driver 15 controls rotating and sliding motions of the C-arm 14. The C-arm driver 15 includes a plurality of power sources for realizing various motions of the C-arm 14. The C-arm driver 15 causes the C-arm 14 to execute various motions in accordance with a drive signal from processing circuitry 44 (drive control function 442) of the console apparatus 40.

The sensor 20 is a pressure detecting sensor, which is assumed to be, for example, a film-type pressure sensitive sensor. The film-type pressure sensitive sensor may be of any type, for example, a strain gauge sensor, a resistive film sensor, a capacitance sensor, or the like. By using the film-type pressure sensitive sensor, the detection surface of the FPD can be nearer to the subject, thereby improving the image quality. Furthermore, by placing the sensor nearer to the subject, the upper limit value of the X-ray dosage can be higher, so that imaging can be performed with a higher dose.

The sensor is not limited to the film-type pressure sensitive sensor, but may be a microswitch-type pressure sensor. The sensor 20 is disposed at a position where a contact needs to be detected; in other words, a position where each of the modules of the X-ray diagnosis apparatus 1, such as the imaging apparatus 10, the table apparatus 30, or the console apparatus 40, may be brought into contact with another object in the room including the subject P. For example, it suffices that the sensor is disposed in an instrument, such as the X-ray generator 12, the X-ray detector 13, the C-arm 14, a housing of the console apparatus 40, the table top 33 of the table apparatus 30, or a support frame 34, included in the X-ray diagnosis apparatus 1.

The table apparatus 30 is a module configured to move the subject P placed thereon. The table apparatus 30 includes a base 31, a table driver 32, the table top 33, the support frame 34, and the sensor 20.

The base 31 is a housing configured to support the support frame 34 in such a manner that the support frame 34 can move vertically (in a Z direction). The base 31 is disposed on a floor surface and houses the table driver 32.

The table driver 32 is a motor or an actuator configured to move the table top 33, on which the subject P is placed. The table driver 32 moves the table top 33 in a horizontal direction (in an X direction and a Y direction) or a vertical direction (the Z direction) relative to the floor surface in response to a drive signal from the processing circuitry 44 (drive control function 442) of the console apparatus 40. Thus, the table driver 32 changes the position of the subject P relative to the imaging direction. Note that the table driver 32 may be configured to move the support frame 34 together with the table top 33 in a longitudinal direction of the table top 33 (in the Y direction).

The table top 33 is a plate on which the subject P is placed. The table top 33 is provided on top of the support frame 34.

The support frame 34 is a frame configured to support the table top 33 to be movable in the longitudinal direction (the Y direction). The support frame 34 is provided on top of the base 31.

In the table apparatus 30, the table top 33 may be of a type in which the table top 33 is movable relative to the support frame 34 (a single-stage sliding type), or a type in which both of the table top 33 and the support frame 34 are slidable relative to the base 31 (a double-stage sliding type).

The console apparatus 40 is a module configured to control the entire operation of the X-ray diagnosis apparatus 1. The console apparatus 40 is configured to execute various controls in response to various input operations from an operator who uses the X-ray diagnosis apparatus 1. The console apparatus 40 is configured separately from the imaging apparatus 10 and the table apparatus 30. The console apparatus 40 or at least a part of the components of the console apparatus 40 may be mounted on the imaging apparatus 10 or the table apparatus 30. The console apparatus 40 of the present embodiment executes a plurality of functions in the single console. Alternatively, the console apparatus 40 may execute a plurality of functions in a plurality of consoles. The console apparatus 40 includes the memory 41, a display 42, the input interface 43, the processing circuitry 44, and the sensor 20.

The memory 41 is a storage device storing various information (e.g., an X-ray image, a program, data, a trained model, a statistic value, and a threshold). The memory 41 is, for example, a hard disk drive (HDD), a solid state drive (SSD), or an integrated circuit (IC). The memory 41 may be a portable storage medium, such as a CD (compact disc), a digital versatile disc (DVD), a flash memory, or a random access memory (RAM). The memory 41 may be a drive device that writes or reads various information to or from the portable storage medium. The storage area of the memory 41 may be in the X-ray diagnosis apparatus 1, or in an external storage device connected to the X-ray diagnosis apparatus via the network.

The X-ray image stored in the memory 41 is a two-dimensional image or a frame image based on two-dimensional X-ray projection data obtained by sequentially storing projection data in units of rows or columns. Programs stored in the memory 41 are executed by, for example, the processing circuitry 44. The programs include a control program for the X-ray diagnosis apparatus 1. Data stored in the memory 41 are, for example, projection data output from the X-ray detector 13 of the imaging apparatus 10, data before processing, during processing, and after processing by the processing circuitry 44, or various tables.

The display 42 is a display device configured to display various information (e.g. X-ray images and graphical user interface (GUI)). The display 42 is, for example, a cathode ray tube (CRT) display or a liquid crystal display (LCD). The display 42 may be a desk-top type or a tablet-type display device connected to the console apparatus 40 in a communicatable manner.

The input interface 43 accepts various input operations from the operator who uses the X-ray diagnosis apparatus 1, converts them to electric signals, and outputs the converted electric signals to the processing circuitry 44. The input interface 43 accepts input operations of subject information, imaging conditions, instructions for moving the C-arm 14 or the table top 33, setting of a region of interest, etc. The input interface 43 comprises a physical operation part (e.g., a mouse, a keyboard, a track ball, a switch, a foot switch, a button, a joystick, a touch pad, and a touch panel display). The input interface 43 may be a circuit configured to accept various input operations from an external input device provided separately from the X-ray diagnosis apparatus 1, convert them to electric signals, and output the converted electric signals to the processing circuitry 44.

The processing circuitry 44 controls the entire operation of the X-ray diagnosis apparatus 1. The processing circuitry 44 includes at least one processor. In the present embodiment, the processing circuitry 44 realizes various functions (e.g., an imaging condition setting function 441, a drive control function 442, a contact detecting function 443, a contact information generating function 444, an image generating function 445, and a display control function 446).

The processing circuitry 44 sets conditions for imaging of an X-ray image relating to the subject P (hereinafter, “imaging conditions”) through the imaging condition setting function 441. The imaging conditions include conditions for generation or emission of an X-ray (hereinafter referred to as X-ray conditions) and conditions for an image quality of an X-ray image taken under a predetermined X-ray condition (hereinafter referred to as image quality conditions).

The X-ray conditions are various parameters relating to setting of a dose of X-rays to be emitted. Specifically, the X-ray conditions include a tube voltage kV, a tube current mA, a product of the tube current and an emission time (tube current-time product) mAs, a pulse width msec, a pulse rate, a type and thickness of a beam filter, a size of an X-ray radiation field, a focal spot size, a dose, etc.

The image quality conditions are various parameters relating to generation of an X-ray images. Specifically, the image quality conditions include a spatial resolution, a binning number, an element size, the number of elements of the FPD included in the X-ray detector 13, and a pixel size and the number of pixels (resolution) of the X-ray image.

The processing circuitry 44 executes control for driving the C-arm driver 15 of the imaging apparatus 10 and the table driver 32 of the table apparatus 30 based on the set imaging conditions through the drive control function 442. Specifically, the processing circuitry 44 generates a drive signal based on information relating to driving of the C-arm driver 15 and the table driver 32 input through the input interface 43. The processing circuitry 44 outputs the generated drive signal to the C-arm driver 15 and the table driver 32, and controls the motions of the C-arm 14 and the table top 33 in real time. Thus, the projection data relating to the subject P is obtained.

The processing circuitry 44 detects contact between an object and either or both of the imaging apparatus 10 and the table apparatus 30 through the contact detecting function 443. For example, the processing circuitry 44 determines whether output value information relating to a degree of contact with the object detected by the sensor 20 through the contact detecting function 443 (e.g. a sensor value) is equal to or greater than a threshold.

The processing circuitry 44 generates, through the contact information generating function 444, contact position information relating to a contact position with respect to the object based on the detection result through the contact detecting function 443. The output value information and the contact position information are also collectively referred to as contact information.

The processing circuitry 44 generates an X-ray image through the image generating function 445. Specifically, the processing circuitry 44 generates an X-ray image using the projection data output from the X-ray detector 13 of the imaging apparatus 10 based on the image quality conditions set through the imaging condition setting function 441. The generated X-ray image is stored in, for example, the memory 41.

Through the display control function 446, the processing circuitry 44 performs a control to cause the contact position information to be displayed on the display 42. The processing circuitry 44 may further cause the output value information to be displayed through the display control function 446 together with the contact position information. In other words, the contact information may be displayed on the display through the display control function 446.

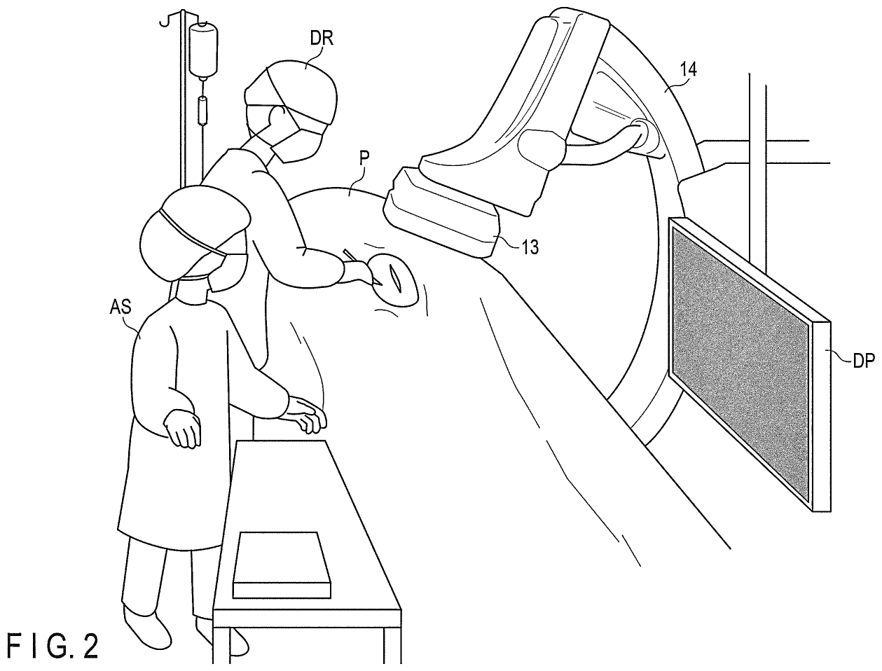

Next, an environment in which the contact detection processing is executed in the X-ray diagnosis apparatus 1 according to the present invention will be described with reference to the conceptual diagram of FIG. 2.

FIG. 2 is a conceptual diagram showing a state of an operation room where the X-ray diagnosis apparatus 1 is disposed. The X-ray detector 13 of the X-ray diagnosis apparatus 1 is pressed against the subject P who is a patient lying on the table top 33 of the table apparatus 30, and an X-ray image is taken. A doctor DR performs together with an assistant AS medical care and an operation for the patient while observing a display DP.

Assuming such a situation, the doctor DR conducts treatment while concentrating on the hands and the display screen of the display DP. At that time, if the imaging apparatus 10 and the table apparatus 30 of the X-ray diagnosis apparatus 1 are moved, these may be brought into contact with various objects in the operation room, including instruments placed near the subject P, such as an injector for injecting a contrast agent, an intravenous drip stand, a cart on which surgery instruments are placed, or the like, other medical instruments such as an electrocardiographic monitor for measuring vital data, health-care professionals, and the like. For example, when the table apparatus 30 is driven, it may be brought into contact with the intravenous drip stand, or the subject P and the X-ray detector 13 may be brought into contact with each other more than necessary. The doctor DR and the assistant AS must pay attention to all of the movement and driving of these instruments and medical instruments. The X-ray diagnosis apparatus 1 according to the present embodiment detects contact between an instrument and an object with a high accuracy, and displays contact information on the display DP, so that an accident due to the contact can be avoided.

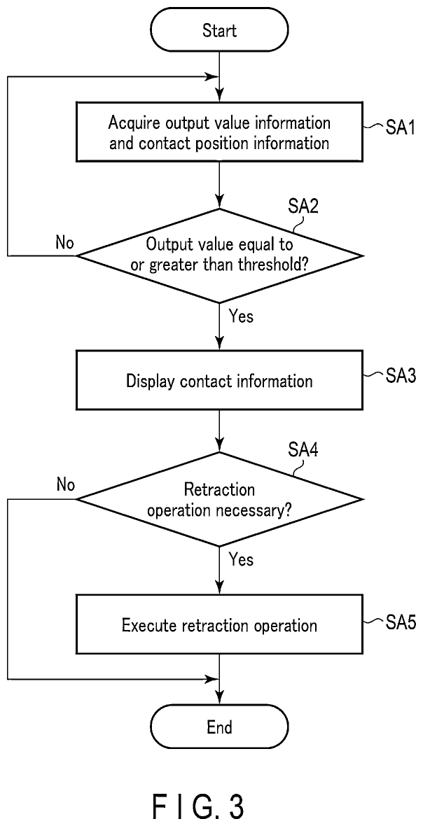

Next, an operation example of the X-ray diagnosis apparatus 1 according to the present embodiment will be described with reference to the flowchart of FIG. 3.

In step SA1, the sensor 20 acquires output value information and contact position information. In the embodiment, the output value information is an output value of the sensor 20 (a sensor value), for example, a pressure. The contact position information includes at least one of an identification number of the sensor 20, position information of the sensor 20 in the room, or sensor surface position information indicating what portion of the sensor 20 contacts another object. In a case where the sensor 20 includes a geomagnetic sensor, a spatial position may be specified from a sensor value of the geomagnetic sensor as the position information of the sensor 20. Alternatively, a table showing the relationship between each sensor 20 and a position of an apparatus where the sensor 20 is attached, in which the identification number of the sensor 20 is associated with an attachment position, may be prepared in advance, and the position information of the sensor 20 may be specified with reference to the table based on the identification number of the sensor 20 from which the output value was acquired.

In step SA2, the processing circuitry 44 determines whether or not the output value is equal to or greater than the threshold, through the contact detecting function 443. If the output value is equal to or greater than the threshold, the processing proceeds to step SA3. If the output value is smaller than the threshold, the processing returns to step SA1 and similar processing is continued.

In step SA3, the processing circuitry 44 causes contact information to be displayed through the display control function 446. The contact information may be displayed on the display DP in the room shown in FIG. 2, or on the display 42 of the console apparatus 40. The contact information includes, for example, output value information and contact position information, and indicates a position of the instrument (or a part of the instrument) that is brought into contact with the sensor, the name of that part, and a contact state. The contact information may be displayed on the display, or reported by voice, such as synthetic voice, together with the display.

In step SA4, through the contact detecting function 443, the processing circuitry 44 determines whether a retraction operation of the instrument is necessary due to the contact. Specifically, the processing circuitry 44 may determine that the retraction operation is necessary, when the contact between the instrument and the object occurs, or if the output value indicated in the output value information is equal to or greater than the threshold. If the retraction operation of the instrument is necessary, the processing proceeds to step SA5. If the retraction operation of the instrument is not necessary, the processing for the acquired output value is ended, and similar processing from step SA1 is repeated with respect to an output value the sensor 20 acquires through the next sensing.

In step SA5, the processing circuitry 44 executes the retraction operation of the instrument through the drive control function 442. The retraction operation is a control of moving the contacted instrument in a separating direction, or a control of stopping the driving of the contacted instrument. For example, the X-ray detector 13 of the imaging apparatus 10 is controlled to move in a direction separating from the subject P.

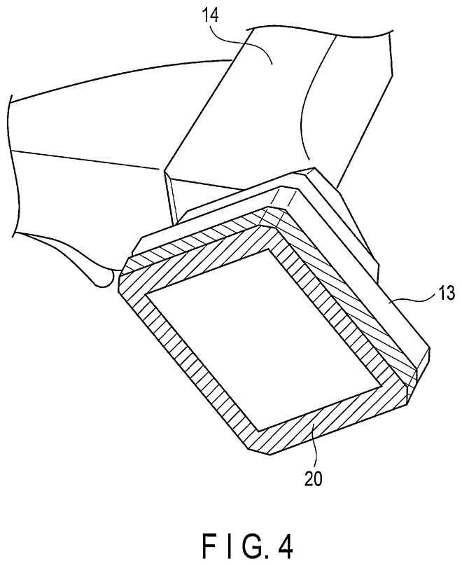

Next, an example of the placement of the sensor 20 will be described with reference to FIG. 4.

FIG. 4 shows an example in which the sensor 20 is placed on the X-ray detector 13. The sensor 20 is placed on a surface of the X-ray detector 13 that faces and is brought into contact with the subject P so as to surround the outer periphery of the surface. The sensor 20 is placed on the overall surface that is brought into contact with the subject P, not only the outer periphery of the surface.

The sensor 20 may be placed to cover the side surfaces of the X-ray detector 13. Although not shown, the sensor 20 may be placed on a back surface of the X-ray detector 13 opposite to the surface that faces the subject P, and a buffer member may be placed on a front surface of the X-ray detector 13. With this configuration, for example, if the X-ray detector 13 is pressed against the subject P, the pressure applied to the subject P can be absorbed. If the X-ray detector 13 collides against another instrument, the shock can also be absorbed.

The buffer member may be placed on the surface (which is brought into contact with the subject) of the sensor 20. The buffer member may also be placed on the back surface of the sensor 20, in which case when the sensor 20 is in contact with the subject, the pressure can be absorbed and dispersed by the buffer member on the back surface.

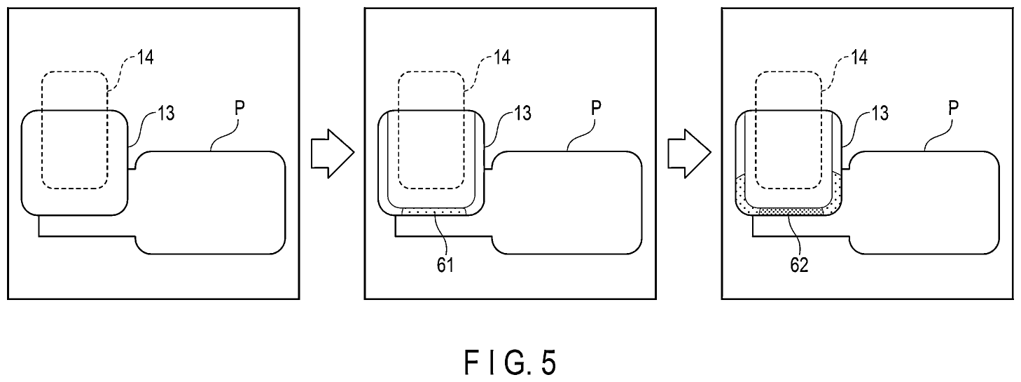

Next, a first display example of contact information displayed on the display DP or the display 42 of the console apparatus 40 will be described with reference to FIG. 5.

FIG. 5 shows a window for displaying contact information, which is indicated on the display in addition to a medical image or a video image captured by a camera (not shown). The window may be indicated at any position of the display, such as a lower right position, a lower left position, or the like. Regarding the timing when the window is displayed, a window related to the sensor 20 that is placed on an instrument or a portion that the health-care professional must pay attention to may be indicated at all times. Alternatively, a window showing contact information related to the sensor 20 may be indicated on the display in a case where the output value acquired by the sensor 20 is equal to or greater than the threshold.

Specifically, FIG. 5 shows a case in which the output value of the sensor 20 placed on the X-ray detector 13 is equal to or greater than the threshold, and schematically shows the C-arm 14, the X-ray detector 13 connected to the C-arm 14, and the subject P in the window.

The window at the left side of FIG. 5 shows a state in which the subject P and the X-ray detector 13 are in contact, but contact information is not indicated since the output value of the sensor 20 has not exceeded the threshold.

The window in the middle of FIG. 5 shows a state in which the subject P and the X-ray detector 13 are in contact. In this state, the output value is equal to or greater than the threshold, and contact information 61 is indicated in a portion where the output value is equal to or greater than the threshold in a manner corresponding to the output value. Specifically, an area of a color different from that of the other areas is indicated in a central portion of the surface of the X-ray detector 13 that is in contact with the subject P. In the example of FIG. 5, the difference in color is expressed by hatching. The area of the different color represents a portion where the contact with the object occurs.

The window at the right side of FIG. 5 shows a state in which the sensor 20 detects a higher pressure than that in the state shown in the middle of FIG. 5, and contact information 62 representing the higher pressure is indicated in a wider range as compared to the contact information 61 shown in the middle of FIG. 5. In this embodiment, it is assumed that the degree of the pressure value is expressed by a difference in color; for example, a low pressure value is indicated by blue, and a high pressure value is indicated by red. In FIG. 5, the difference in color is expressed by a difference in density of hatching. The higher the pressure value in the contact information, the higher the density of the hatching.

As described above, the color of the contact position in the sensor 20 is changed in accordance with the output value (pressure value) due to the contact with an object as in the states from the left side to the right side of FIG. 5, so that the health-care professional can easily perceive the position where the contact occurs and the pressure value at that position. A numerical value may be indicated as the contact information in addition to the color.

Distinction between the contact information 61 and the contact information 62 in FIG. 5 is not limited to the difference in color, but may be represented by a gray scale, or a mark such as an arrow. For example, the contact position may be represented by a position of the arrow, and the output value may be represented by a size of the arrow. Furthermore, in the case of the X-ray detector 13, the indication of the contact information may be changed in accordance with the position where the sensor 20 is placed; for example, a pressure detected by the sensor 20 placed on the front surface of the X-ray detector 13 may be indicated by red, and a pressure detected by the sensor 20 placed on a side surface of the X-ray detector 13 may be indicated by green. The indication of the contact information is not limited to the above. The contact information may be displayed in any other manner as long as the contact position and the pressure value at that position can be discriminated.

Assuming that the X-ray detector 13 is brought into contact with the subject P, for example, when the X-ray detector 13 is pressed against the cranial bone, the subject P will feel pain even with a low pressure, since fat beneath the skin is thin in the head. In contrast, since the abdomen has thicker fat beneath the skin and a greater amount of muscle as compared to the head, the subject P tends to feel less pain even if the contact pressure is relatively high. Therefore, the sensitivity at which the sensor 20 detects the contact with the subject P may be changed in accordance with the contact region of the subject P where the sensor 20 is in contact with it.

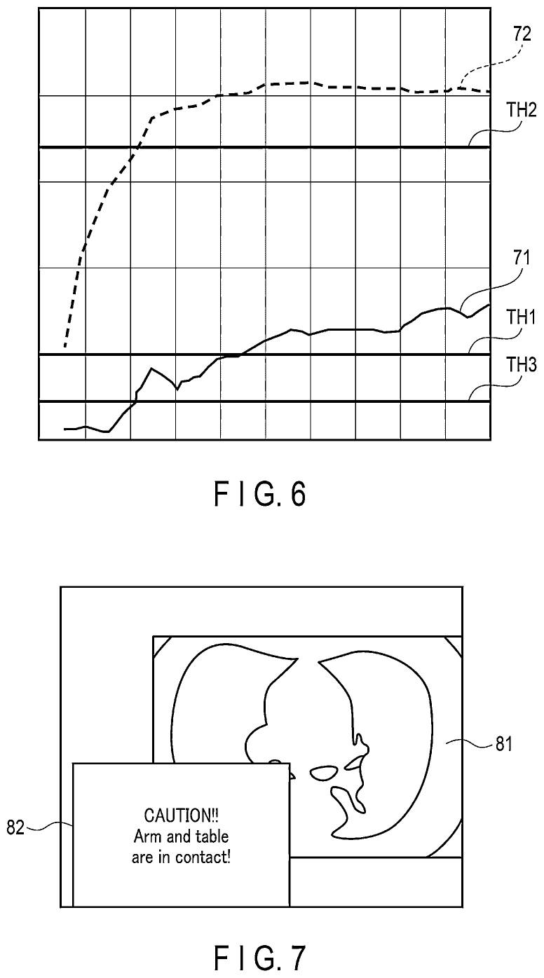

A case of changing the sensitivity in accordance with the contact region will be described with reference to FIG. 6.

FIG. 6 shows time-series data of sensor values acquired by the sensor 20. Graph 71 represents data in a case where the contact region is the head, and graph 72 represents data in a case where the contact area is the abdomen.

Specifically, a threshold table in which a contact target region (region of interest: ROI), such as the head and the abdomen, and a threshold are associated is prepared in advance, and stored in, for example, the memory 41. Through the contact detecting function 443, the processing circuitry 44 extracts a threshold corresponding to a contact target region acquired from order information with reference to the threshold table, and uses the extracted threshold as the threshold for detecting contact.

Referring to FIG. 6, through the contact detecting function 443, the processing circuitry 44 sets a first threshold TH1 in the case where the region of interest is the head and a second threshold TH2 greater than the first threshold TH1 in the case where the region of interest is the abdomen. An appropriate sensitivity can be set in this manner.

Furthermore, in a case where a film-type pressure sensitive sensor is used as the sensor 20, since the FPD of the X-ray detector 13 is capped with a sterilizing cap when used, a very low pressure due to slight contact with the sterilizing cap may be detected by the film-type pressure sensitive sensor. Therefore, as shown in FIG. 6, a lower limit value (a third threshold TH3 in this embodiment) is set, so that a sensor value equal to or smaller than the lower limit value may not be detected as a contact state. In other words, even if a sensor value equal to or smaller than the lower limit value is detected, contact information is not displayed. Accordingly, erroneous detection due to a very slight contact state is avoided, and the contact to be detected can be correctly detected. The lower limit value may be set to the same value as the threshold (TH1 or TH2) to detect contact.

In addition, a display threshold may be set as a threshold lower than the thresholds for detecting contact (the second threshold TH2 in the case of the abdomen or the first threshold TH1 in the case of the head). For example, a fourth threshold lower than the threshold TH1 but greater than the lower limit value is set. If the processing circuitry 44 detects a sensor value equal to or greater than the fourth threshold through the contact detecting function 443, the processing circuitry 44 determines that the value may be determined as contact depending on a subsequent driving. Through the display control function 446, the processing circuitry 44 may display a screen showing a contact position without output value information, for example, as shown in the left side of FIG. 5. As a result, the health-care professional can be notified of a situation in which contact may occur even when the X-ray diagnosis apparatus 1 has not determined to be in “contact”. Therefore, the health-care professional can preliminarily recognize a possibility of contact with the subject or interference between instruments. Thus, the X-ray diagnosis apparatus 1 can support the health-care professional to avoid collision or interference in advance.

Next, a second display example of contact information displayed on the display DP or the display 42 of the console apparatus 40 will be described with reference to FIG. 7.

FIG. 7 shows an example in which contacted instruments are popped up as textual information. If the sensor 20 detects contact in a state in which a medical image 81 is indicated in the display, textual information “CAUTION!! Arm and table are in contact!” is displayed in a pop-up window 82. In this embodiment, if the C-arm 14 and the table apparatus 30 (for example, a front end of the table top 33) interfere with each other, the contact information indicating the interference between the arm and the table is displayed as a pop-up, so that a health-care professional, such as a doctor or a nurse, can immediately perceive the interference.

According to the present embodiment, a plurality of pressure sensors are placed in a plurality of positions of the imaging apparatus and the table apparatus of the X-ray diagnosis apparatus, and it is determined through the contact detecting function that contact has occurred in a case where the output value of a pressure sensor is equal to or greater than the threshold. The display control function causes the display to display contact position information relating to the position where contact occurred and output value information relating to the output value of the sensor. As a result, contact with an object can be detected with a high accuracy. Furthermore, due to the display of the contact position information and the output value information on the display, the health-care professional can perceive he contact position and the degree of pressure of the contact. Accordingly, it is possible to prevent an accident due to the interference and contact between instruments or between an instrument and the subject.

The term “processor” used in the above explanation means, for example, circuitry such as a CPU (central processing unit), a GPU (graphics processing unit), an ASIC (application specific integrated circuit), or a programmable logic device (for example, a simple programmable logic device (SPLD), a complex programmable logic device (CPLD), or a field programmable gate array (FPGA)). If the processor is, for example, a CPU, the processor realizes its function by reading and executing the program stored in the storage circuitry. On the other hand, if the processor is, for example, an ASIC, the function corresponding to a program is directly incorporated into a circuit of the processor as a logic circuit, instead of being stored in the storage circuitry. The processors described in connection with the above embodiment are not limited to single-circuit processors; a plurality of independent circuits may be integrated into a single processor that realizes the functions. In addition, a plurality of structural elements shown in the drawings may be integrated into one processor to realize the functions of the structural elements.

According to the embodiment described above, contact with an object can be detected with a high accuracy.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray diagnosis apparatus comprising:
a table including a table top on which a subject is placed;
an imaging unit including an X-ray tube which emits X-rays to the subject and an X-ray detector which detects the X-rays; and
processing circuitry configured to:
detect contact between an object and at least one of the imaging unit and the table; and
execute control to display contact position information relating to a position in contact with the object based on a detection result.

2. The X-ray diagnosis apparatus according to claim 1, wherein the processing circuitry executes control to further display output value information relating to a degree of contact with the object.

3. The X-ray diagnosis apparatus according to claim 2, wherein the processing circuitry executes control to display the contact position information or the output value information in a different display manner in accordance with at least one of a position indicated by the contact position information and a magnitude of an output value indicated by the output value information.

4. The X-ray diagnosis apparatus according to claim 2, wherein the processing circuitry executes control to display the output value information in different colors in accordance with magnitudes of the output values indicated by the output value information.

5. The X-ray diagnosis apparatus according to claim 1, wherein the processing circuitry sets a lower limit value to detect the contact in accordance with each contact region of the subject.

6. The X-ray diagnosis apparatus according to claim 1, wherein the processing circuitry is further configured to execute a retraction operation for retracting an instrument relating to the contact, in a case where an output value indicated by the output value information relating to a degree of contact with the object is equal to or greater than a threshold.

7. The X-ray diagnosis apparatus according to claim 1, wherein the processing circuitry executes control not to display the contact position information, in a case where an output value indicated by the output value information relating to a degree of contact with the object is smaller than a threshold.

8. The X-ray diagnosis apparatus according to claim 1, wherein the processing circuitry executes control to display the contact position information, in a case where an output value indicated by the output value information relating to a degree of contact with the object is smaller than a first threshold and equal to or greater than a second threshold that is smaller than the first threshold.

9. The X-ray diagnosis apparatus according to claim 1, further comprising a pressure sensor placed in a position where a contact may occur among the table, the imaging unit, and the object.

10. The X-ray diagnosis apparatus according to claim 1, further comprising a sensor placed on at least one of a side surface of the X-ray detector, an outer periphery of a first surface of the X-ray detector that faces the subject, and a back surface of the first surface.
